(19) United States
(12) Patent Application Publication
(10) Pub. No.: US 2006/0088833 A1
(43) Pub. Date: Apr. 27, 2006

(54) Detection of mycobacteria in clinical material

(76) Inventors: Franz-Christoph Bange, HANNOVER (DE); Erik Christian Bottger, Zurich (CH)

Correspondence Address: OSTROLENK FABER GERB & SOFFEN, 1180 AVENUE OF THE AMERICAS, NEW YORK, NY, 100368403, US

(21) Appl. No.: 10/510,329

(22) PCT Filed: Apr. 4, 2003
(86) PCT No.: PCT/EP03/03533

§ 371 Date: Nov. 11, 2005

(30) Foreign Application Priority Data
Apr. 6, 2002 (DE) 102 15 238.1

(51) Int. Cl. C12Q 1/68; C07H 21/04

(52) U.S. Cl. 435/6; 536/24.1

(57) ABSTRACT

The present invention relates to a molecular biological method for the specific detection of mycobacteria and for the differentiation of the Mycobacterium tuberculosis complex and Mycobacterium avium from other mycobacteria in clinical material by means of amplification primers and oligonucleotide probes which are specific for the genus- and species-specific regions of the 16S rRNA gene of mycobacteria.

The present invention relates to a method for the specific detection of mycobacteria and for the differentiation of the Mycobacterium tuberculosis complex and Mycobacterium avium from other mycobacteria in clinical material.

Tuberculosis is an infectious disease which is widespread around the world, has a chronic course and each year leads to the death of more people than any other bacterial infection.

Tuberculosis is particularly localized in the lung, and less commonly in the cervical lymph nodes, bowel or skin. The great differences in the clinical course of tuberculosis therefore make accurate description of the pathological state and rapid diagnosis, especially at early stages of the disease, necessary.

The species which cause tuberculosis are: Mycobacterium tuberculosis and, very rarely, Mycobacterium bovis. These causes of tuberculosis are generally comprehended by the term “Mycobacterium tuberculosis complex”.

One example of the sequelae of chronic tuberculosis, which is induced in particular owing to the spread of the causes of tuberculosis throughout the body, is tuberculous meningitis, which can be diagnosed by lumbar puncture, in which case unambiguous and early diagnosis and subsequent targeted intensive therapy alone are life-saving. Further sequelae can likewise be identified or prevented only by unambiguous and early diagnosis: tuberculons pleurisy, tuberculons peritonitis, tuberculosis of skin, tuberculosis of bones, tuberculosis of joints and tuberculosis of the genitourinary system. Tuberculosis of the genitourinary system in particular has an insidious course with few symptoms and therefore often cannot be immediately recognized as such, which is why unambiguous and early diagnosis of patients is necessary.

In most industrialized countries, immediate notification of a case of the disease is obligatory, not least in order to prevent as quickly as possible any spread in the population. In some developing countries such as Africa, Asia or Oceanea the average incidence is 200 new cases of the disease per 100,000 population per year. The average incidence even in Western Europe is 30 new cases of the disease per 100,000 population per year, and it is about 20 in the Federal Republic of Germany.

An increased occurrence of tuberculosis has been observable in recent years both in the developing countries and in the industrialized nations, deaths being recorded regularly in particular in immunosuppressed HIV patients (8).

It is assumed that at present one third of the world's population is infected by the Mycobacterium tuberculosis complex. However, there is a demand for rapid and unambiguous diagnosis to detect these bacterial strains (9, 14), not just because of the general risk for large parts of the world's population, but also because of the occurrence now resembling an epidemic of multidrug-resistant bacterial strains (MDRTB) inside or outside hospitals.

The HIV epidemic in recent decades, acting as “nutrient medium” for the spread of infectious diseases, has also led in addition to the increased epidemiological occurrence of non-tuberculous mycobacteria (4). Infections caused by non-tuberculous mycobacteria resemble a “genuine” tuberculosis in affecting organs such as lung, lymph nodes in the neck region or the skin. On rare occasions there is dissemination of the non-tuberculous pathogens throughout the body, in which case sequelae resembling those of a “genuine” tuberculosis may be observed. Non-tuberculous mycobacteria lead in patients suffering for example from cystic fibrosis to an additional deterioration in the pathological state in the region of the lung (1, 2, 16).

The non-tuberculous mycobacteria observed in clinical practice include: Mycobacterium avium, Mycobacterium intracellulare, Mycobacterium kansasii, Mycobacterium marinum, Mycobacterium fortuitum, Mycobacterium chelonae and Mycobacterium abscessus (4).

The diagnosis of mycobacteria in clinical material should ideally permit specific detection of tuberculous and of non-tuberculous mycobacteria.

In recent years there has been increased use of techniques of molecular biology based on nucleic acid multiplication, in particular amplification, such as the polymerase chain reaction (PCR), for the clinical diagnosis of mycobacteria in the laboratory. PCR methods for amplifying a large number of different chromosomal DNA fragments have been employed to detect both genus-specific and M. tuberculosis-specific DNA regions (15).

Genus-specific methods are employed to distinguish a mycobacterial infection from other infections. Known genus-specific methods are targeted on the 16S rRNA gene or the gene of the 65 kDa—“heat shock” protein. Subsequent specific identification of some mycobacterial species is carried out using conserved hybridization probes (5, 12). Alternatively, the amplified genes are sequenced (6) or a restriction enzyme analysis is carried out (15).

The specific DNA regions employed for the species-specific detection of the M. tuberculosis complex by means of PCR include, for example, IS 6110, the genes of the 38 kDa-protein, the genes of the MBP 64-protein, and the regions mtp4o or pMTb4.

Apart from these individual laboratory methods, there are commercially available diagnostic kits which are mostly suitable exclusively for the diagnosis of the M. tuberculosis complex. Known kits are supplied for example by Roche-Amplicor™, Geneprobe™ or Abbot™.

The methods of molecular biology carried out for this purpose include on the one hand multiplication of the nucleic acid to be detected (target amplification), for example by the PCR, by the transcription-based isothermal DNA synthesis (TMA), by the ligase chain reaction (LCR) or by the isothermal strand displacement amplification (SDA), and on the other hand multiplication of the signal-emitting component (signal amplification), such as by isothermal Qβ replication.

The significant fact in the current situation is that there is as yet no method accepted as generally acknowledged diagnostic standard. In Germany, the detection of mycobacteria with the aid of molecular methods is now monitored twice a year by a national quality control. These interlaboratory studies revealed weaknesses in the commercially available diagnostic kits and in various independent developments, especially when applied to highly diluted samples. It is assumed that systematic weaknesses of the respective underlying detection reactions are involved. Therefore, all the currently existing methods are regarded as unsatisfactory and thus in need of improvement.

Further considerable disadvantages of the commercially available methods are in particular (a) no possibility of differentiating between the Mycobacterium tuberculosis complex and non-tuberculous mycobacteria, (b) a possible nonspecific inhibition of the amplification reaction in the detection method with clinical samples, (c) investigation takes a long time (several hours) and (d) the clinical material which can be employed is restricted in particular to samples from the respiratory tract.

The 16S rRNA gene is already employed for the detection and identification of various human-pathogenic mycobacteria by PCR (7). Based on this 16S rRNA detection system, an algorithm is proposed for the specific multiplication of a 1000 bp fragment of the mycobacterial 16S rRNA using a nonspecific primer and a genus-specific primer for mycobacteria. To confirm that the correct fragment is multiplied, genus-specific oligonucleotide probes which hybridize with the amplified fragment are employed. Species-specific hybridization probes are employed subsequently, it being possible by means of the same amplified fragment to differentiate the mycobacterial species M. tuberculosis complex and M. avium from other bacterial species (5). The substantial disadvantages of these multistage methods are that these detection methods are based on hybridization methods which are complicated in part, and requires the employment of highly qualified people. In addition, these detection methods must be set up at least overnight; a result is therefore available at the earliest on the day following provision of the sample (5, 6). It is usually necessary for patients to be admitted to hospital until the results of the investigation are available. These methods can therefore be employed only with many provisos for routine clinical diagnosis.

Up until now no method is known in the art which is suitable for routine clinical use and is distinguished in particular by simple application, and with which it is possible to detect M. tuberculosis and M. avium faster and specifically in clinical material such as sputum, bronchial lavage, gastric juice, urine, stool, bone marrow, blood or biopsies, in particular puncture biopsies, and in the same method unambiguously to distinguish mycobacterial infection from other microbial infections.

DNA extracted from clinical samples contains impurities which often lead to inhibition of enzyme-based amplification, in particular the PCR. Thus, with the detection methods known at present, there is a great risk that a negative result will be obtained although the patient in fact has a mycobacterial infection. In the worst case, this results in an existing tuberculosis being overlooked. There has also therefore been the need for some time to develop a control system which precludes so-called “false-negative” findings in the detection method (inhibition control).

It is known that the use of real-time PCR (Rapid Cycle PCR), which is equipped for example with an air temperature-controlled system and thus exhibits considerably shorter transition times compared with a conventional PCR, leads to a distinctly reduced time until, for example, M. tuberculosis is detected (2).

In addition, fluorimetric measurements, especially when they are employed within the framework of the real-time PCR method, represent a fast and sensitive method for detecting amplified gene fragments. In (3), a real-time fluorimetry was employed to detect M. tuberculosis in expectorations using the TaqMan™ system.

The LightCycler™ system of Roche Molecular Biochemicals, which is an embodiment of real-time PCR, has now likewise been employed to detect M. bovis in bovine faeces and to detect rifampin or isoniazid resistence-related mutations in M. tuberculosis (11, 13). In both these studies, the multiplied fragments were typically 200 base pairs long. It has to date been assumed, because of the high throughput of the LightCycler™ system, that multiplication of larger DNA fragments of mycobacteria is difficult or impossible because of the high CG nucleotide content of about 65% up to 75% which occurs in mycobacteria.

Based on the prior art, a considerable disadvantage of previously disclosed detection reactions is that mycobacterial infections in clinical material cannot yet be distinguished unambiguously from non-mycobacterial infections: the genus-specific region II, “genus II”, on the 16S rRNA gene of mycobacteria is known in the art, it being possible by means of a specific hybridization probe pair and of a melting curve analysis to differentiate a large number of mycobacterial species on the basis of a higher melting point of the probe pair from other bacterial species and other microorganisms (6). However, certain mycobacterial species such as M. triviale, M. agri, M. Xenopi or M. chitae also show a low melting point, so that it is quite impossible according to the state of the art to distinguish mycobacteria from non-mycobacteria unambiguously, especially in a single method step.

With this background, the technical problem of the present invention is to provide an improved method which enables in particular a particularly fast and, at the same time, more specific detection of mycobacterial infections in clinical material of varying origin and identification of the species M. tuberculosis complex and/or M. avium in a joint detection method.

The present invention solves the technical problem by providing a method for the joint, specific detection of a mycobacterial infection and of the Mycobacterium tuberculosis complex and/or of Mycobacterium avium vis-à-vis other mycobacterial species in clinical material, where
(a) microbial DNA is extracted from the clinical material and then
(b) at least one fragment of the 16S rRNA gene from the microbial DNA is amplified with a primer pair comprising the nucleotide sequences SEQ ID NO: 1/SEQ ID NO: 5, or
   is amplified with two primer pairs, where one primer pair comprises the nucleotide sequences SEQ ID NO: 2/SEQ ID NO: 3, and the other primer pair is employed immediately previously or subsequently or simultaneously and comprises the nucleotide sequences SEQ ID NO: 4/SEQ ID NO: 5, and then
(c) the at least one amplified 16S rRNA gene fragment is detected by means of at least one pair of labelled hybridization probes which hybridize with hypervariable species-specific regions of the 16S rRNA fragment of mycobacteria, where the pair of labelled hybridization probes for detecting the M. tuberculosis complex comprises the nucleotide sequences SEQ ID NO: 6/SEQ ID NO: 7 or the pair of complementary sequences thereof, and where the pair of labelled hybridization probes for detecting M. avium comprises the nucleotide sequences of SEQ ID NO: 8/SEQ ID NO: 9 or the pair of complementary sequences thereof, and then, simultaneously or immediately previously in time
(d) the at least one amplified 16S rRNA gene fragment is detected by means of a pair of labelled hybridization probes which hybridizes with the genus-specific region III of the 16S rRNA fragment, where the pair comprises the nucleotide sequences SEQ ID NO: 10/SEQ ID NO: 11 or the pair of complementary sequences thereof, and where
(e) the specific detection of the mycobacterial genus and the detection of the M. tuberculosis complex and/or of M. avium takes place in steps (c) and (d) by means of melting curve analysis.

The invention thus advantageously provides for the detection, in a unitary joint procedure, of mycobacteria as genus together with the species-specific detection of M. tuberculosis complex to be possible. The invention also makes a joint unitary specific detection of the genus Mycobacterium together with the species-specific detection of M. avium possible. Finally, the invention also makes the joint specific detection of bacteria of the genus Mycobacterium together with the species-specific detection of M. tuberculosis complex and of M. avium possible.

A considerable advantage of the method according to the invention is that it is possible in the combined detection method employed for the diagnosis of mycobacterial infections to identify, unambiguously and reliably, both an existing mycobacterial infection vis-à-vis other microbial infection, and an existing tuberculosis vis-à-vis non-tuberculous infections. This detection was not possible in the prior art in such an advantageous manner.

The unambiguous detection of a mycobacterial infection vis-à-vis other microbial infections takes place according to the invention by analyzing the melting temperatures of the hybridization of the genus-specific hybridization probe pair, which comprises the nucleotide sequences SEQ ID NO: 10/SEQ ID NO: 11 or the pair of complementary sequences thereof, with the genus-specific region III of the 16S rRNA gene. In the detection methods of the invention, mycobacterial strains are distinguished by melting temperatures of at least 55° C., in particular of 55° C. and 61.5° C. In a further variant of this detection method, when the melting temperature of 55° C. occurs it is possible to identify the mycobacterial strain M. chelonae unambiguously vis-à-vis all other mycobacterial strains, which have in particular a melting temperature of 61.5° C., and to detect a mycobacterial infection by M. chelonae.

As a further advantage, the method of the invention permits not only unambiguous and reliable detection of a tuberculous infection but also unambiguous and reliable detection of a non-tuberculous infection caused by M. avium.

Unambiguous detection of a tuberculous infection by strains of the M. tuberculosis complex takes place according to the invention by analyzing the melting temperatures of the hybridization of the species-specific hybridization probe pair for the M. tuberculosis complex, the pair comprising the nucleotide sequences SEQ ID NO: 6/SEQ ID NO: 7 or the pair of complementary sequences thereof, with the species-specific region of the 16S rRNA gene. In the detection methods of the invention, strains of the M. tuberculosis complex are distinguished by melting temperatures of at least 55° C., in particular of 64° C.

Unambiguous detection of a tuberculous infection by M. avium takes place according to the invention by analyzing the melting temperatures of the hybridization of the species-specific hybridization probe pair for M. avium, which comprises the nucleotide sequences SEQ ID NO: 8/SEQ ID NO: 9 or the pair of complementary sequences thereof, with the species-specific region of the 16S rRNA gene. In the detection methods of the invention, M. avium is distinguished by melting temperatures of at least 55° C., in particular of 61° C.

The aforementioned method is preferably carried out according to the invention with an internal standard of the invention in the form of artificial plasmids, control plasmids, for identifying so-called “false-negative” findings. One variant provides for a first portion of the extracted microbial DNA to be subjected to the aforementioned method with steps (a) to (e), and for a second portion of the extracted microbial DNA to be, in an approach parallel to the aforementioned method,
(a′) mixed with at least one artificial plasmid, preferably subcloned in pGEM-T, which serves as internal standard, where the artificial plasmid includes a genus-specific region III of the 16S rRNA with a modified nucleotide sequence, and then
(b′) fragments of the modified 16S rRNA genes to be multiplied by means of at least one primer pair selected from the group of primer pairs consisting of nucleotide sequence pairs SEQ ID NO: 1/SEQ ID NO: 5 and SEQ ID NO: 4/SEQ ID NO: 5, and then
(c′) the multiplied 16S rRNA fragments to be detected by means of a pair of labelled hybridization probes which hybridize with the modified genus-specific region III, where the pair of labelled hybridization probes comprise the nucleotide sequences SEQ ID NO: 10/SEQ ID NO: 11, and where
(d′) during the detection the 16S rRNA fragments of mycobacteria and the modified 16S rRNA fragments of the internal standard are specifically detected by means of melting curve analysis.

In a further preferred variant of the method of the invention, steps (a), (b), (c), (d) and (e) of the aforementioned method are carried out, where the at least one artificial plasmid is mixed in step (a) with all of the extracted microbial DNA and, after amplification as claimed in step (b), the detection and melting curve analysis for detection of the multiplied 16S rRNA fragments of mycobacteria and of the modified 16S rRNA fragments of the internal standard is carried out, in particular simultaneously, as claimed in steps (c′) and (d′).

Use of a control plasmid in the detection method of the invention makes it advantageously possible to check the success of the amplification reaction even during the mycobacterial detection reaction in order thus to obtain a reliable and unambiguous result particularly quickly. In particular, so-called “false-negative” findings associated with inhibition of amplification are virtually precluded through the use of the plasmid of the invention as internal standard. The specificity and selectivity of the method of the invention are thus distinctly increased compared with the prior art.

In connection with the present invention, the words “primer pair comprising or including the nucleotide sequences”, “a pair of hybridization probes comprising or including the nucleotide sequences” or the like mean that the respective nucleotide sequences or the pair of nucleotide sequences each have/has the nucleotide sequences referred to, meaning that these nucleotide sequences or the pair thereof consist/consists of the specifically mentioned nucleotide sequence alone, or, where appropriate, include/includes further sequences.

In connection with the present invention, the term “Mycobacterium tuberculosis complex” means the tuberculous mycobacterial species Mycobacterium tuberculosis, especially the strain H37Rv, and Mycobacterium bovis, especially the strain R99, these strains being causes of the disease tuberculosis, and the BCG Pasteur strain of Mycobacterium bovis.

In connection with the present invention, the term “tuberculous” describes a property relating to Mycobacterium tuberculosis, especially the strain H37Rv, and Mycobacterium bovis, especially the strain R99, in the narrower sense, and to the BCG Pasteur strain of Mycobacterium bovis in the wider sense, and the pathological state of tuberculosis.

In connection with the present invention, the term “Mycobacterium avium” means the non-tuberculous mycobacterial species Mycobacterium avium, especially the strain ATCC35712, and the subspecies thereof Mycobacterium paratuberculosis, especially the strain Pat.6783.

In connection with the present invention, the term “non-tuberculous” means a property connected with a disease other than tuberculosis and being in particular a mycobacteriosis.

In connection with the present invention, clinical material means clinical samples such as sputum, bronchial lavage, gastric juice, urine, stool, CSF, bone marrow, blood or biopsies, especially puncture biopsies, for example from cervical lymph nodes.

In connection with the present invention, the term “modified nucleotide sequence” means a nucleic acid sequence which differs through exchange, inversion, deletion or addition of at least one nucleotide, as well as an unusual or synthetic nucleotide, from its original sequence, i.e. the wild-type sequence, in at least one nucleotide, preferably in two nucleotides. In this connection, the term “modified” means a property relating to a “modified nucleotide sequence”.

In a preferred embodiment of the aforementioned methods, the amplification of the gene fragments of the 16S rRNA gene is carried out by means of a polymerase chain reaction (PCR). The amplification is preferably carried out by means of real-time PCR (rapid-cycle PCR). It is possible in real-time PCR methods to observe the multiplication of the PCR products in real time amplification cycle by amplification cycle. The amplification is particularly preferably carried out in a LightCycler™ system from Roche Molecular Biochemicals, which is an embodiment of real-time PCR.

For this purpose, in particular, besides the polymerase, the nucleotides, the buffer solutions and the primers, also added to the initial PCR mixture are hybridization probes which bind specifically to the desired PCR amplification products. In this connection, in particular, two sequence-specific oligonucleotide probes labelled with different dyes are used. The sequences of the labelled hybridization probe pairs of the invention are selected so that they hybridize onto the target sequences of the amplified DNA fragment in such a way that, in particular, the 3′ end of one probe is located close to the 5′ end of the other probe, thus bringing the two dyes into the direct vicinity of one another, with the distance between the two probes being in particular between 1 and 5 nucleotides. There is in particular a fluorescence resonance energy transfer (FRET) between the two dyes of the hybridization probes and thus a shift in the fluorescence spectrum, with the degree of fluorescence in this wavelength range being a function of the amount of detected DNA.

The FRET system provides according to the invention for quantitative measurements of the amount of amplified DNA fragments. The selected hybridization probes of the invention are able to bind quantitatively, that is to say stoichiometrically, to the amplified fragments. In this connection, quantitative hybridization depends in particular on the temperature and the degree of homology of the employed oligonucleotide probes with the detected sequence on the amplified fragment.

In a preferred embodiment, the aforementioned fluorimetric detection of specific DNA sequences in the amplified fragments is carried out after amplification of the fragments by means of conventional PCR. In a particularly preferred embodiment, the fluorimetric detection is carried out in a real-time PCR during the amplification reactions, whereby for example the increase in produced DNA as an increase in the fluorescence signal can be followed.

In a preferred embodiment of the aforementioned process of the invention, the specific detection of species-specific and/or genus-specific regions in the amplified DNA fragment takes place after completion of the amplification reaction, where hybridization of the hybridization probe pair, preferably of a FRET pair, onto the regions to be detected is followed by changing, preferably continuously increasing, the temperature within the framework of a melting curve analysis, and simultaneously measuring the fluorescence emitted as a function of the temperature. A melting temperature at which the hybridization probes, in particular the employed FRET pair, now no longer hybridize onto the region to be detected of the amplified DNA fragment is determined in this way. The essential aspect of a melting curve analysis is that the measured melting point is reduced if mismatches occur between the employed hybridization probe pair and the target region on the amplified DNA fragment. There is identification in this way according to the invention, using hybridization probes, in particular using a FRET pair, of the regions of DNA fragments whose sequences differ from one another in the nucleotide sequence only slightly, in particular by one or a few point mutations.

The 16S rRNA gene of mycobacteria advantageously has both conserved and highly variable regions, which permits for example genus-specific amplification of DNA fragments by means of genus-specific primers. The aforementioned methods of fluorescence detection in conjunction with the melting curve analysis are therefore employed according to the invention for the specific detection of the genus-specific region III and of the hypervariable species-specific regions of the M. tuberculosis complex and M. avium on the 16S rRNA gene.

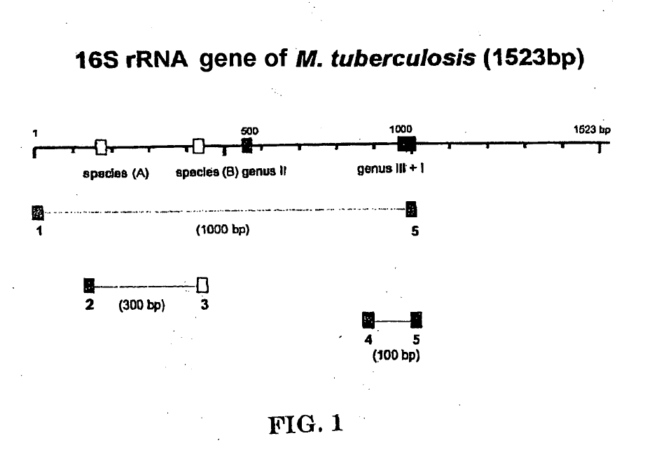

It is known in the art that mycobacterial 16S rRNA genes each comprise two species-specific and two genus-specific regions, see FIG. 1: ‘species (A)’ and ‘species (B)’, respectively ‘genus II’ and ‘genus I’. Within the scope of the present invention, a third genus-specific region on the 16S rRNA gene is described, see FIG. 1: ‘genus III’ (of the invention).

FIG. 1 additionally depicts diagrammatically the primer pairs used for amplification of the selected species-specific and genus-specific regions.

The primer pair including the nucleotide sequences SEQ ID NO: 1 and SEQ ID NO: 5 is employed according to the invention for amplification of the genus-specific region III of 16S rRNA gene of mycobacteria. In a preferred variant, the primer pair consists of degenerate or mutated sequences or fragments thereof, each of which hybridize with the nucleotide sequences SEQ ID NO: 1 and SEQ ID NO: 5, from which they are derived, the degree of homology being in each case at least 90%, preferably at least 95%, particularly preferably at least 98%.

The 1000 bp-long fragment amplified with the aforementioned primer pair comprises both the conserved genus-specific region III and the highly variable species-specific regions of the M. tuberculosis complex and M. avium.

It has particularly surprisingly been found with the method of the invention that efficient amplification of this 1000 bp fragment and detection according to the invention of the genus- and species-specific regions on this fragment was possible in particular using the LightCycler™ system—in contrast to conventional opinion—although this mycobacterial fragment has a high GC content. It is therefore possible and preferred according to the invention to carry out, by means of a single amplified fragment of the 16S rRNA gene of mycobacteria, a genus-specific detection of a mycobacterial infection vis-à-vis other microbial infections and the species-specific detection of the M. tuberculosis complex and M. avium.

The invention moreover provides for the additional or alternative use of two further primer pairs, where one primer pair amplifies an in particular 300 bp-long fragment of the 16S rRNA gene of mycobacteria which comprises the species-specific regions of the M. tuberculosis complex and M. avium, where this primer pair consists of the nucleotide sequences SEQ ID NO: 2/SEQ ID NO: 3, and the second primer pair amplifies a 100 bp-long fragment of the same gene, which comprises the genus-specific region III, where this primer pair includes the nucleotide sequences SEQ ID NO: 4/SEQ ID NO: 5. In a preferred variant, the two primer pairs consist of the pairs of degenerate or mutated sequences or fragments thereof, each of which hybridize with the nucleotide sequence pairs SEQ ID NO: 2/SEQ ID NO: 3 and SEQ ID NO: 4/SEQ ID NO: 5, from which they are derived, the degree of homology being in each case at least 90%, preferably at least 95%, particularly preferably at least 98%.

A pair of labelled hybridization probes which hybridizes with the conserved genus-specific region III is employed according to the invention for detecting the aforementioned amplified 16S rRNA fragments of mycobacteria which comprise the genus-specific region III and/or the species-specific regions of the M. tuberculosis complex and M. avium, where this pair comprises the nucleotide sequences SEQ ID NO: 10/SEQ ID NO: 11 or the complementary sequences thereof. It is preferred in this connection for the hybridization probe pair to be embodied as FRET pair, where the hybridization probe with the nucleotide sequence SEQ ID NO: 10 or the complementary sequence thereof is embodied as donor component (=anchor probe) which is preferably associated at the 3′-terminal nucleotide with a dye, preferably with a fluorescent dye, particularly preferably with fluorescein, and the second hybridization probe consisting of the nucleotide sequence SEQ ID NO: 11 or the complementary sequence thereof is embodied as acceptor component (=sensor probe) which is preferably associated at the 5′-terminal nucleotide with a further dye, preferably with a rhodamine derivative.

Detection of the aforementioned 16S rRNA fragments comprising the species-specific regions is carried out according to the invention using at least one pair of labelled hybridization probes, where the labelled hybridization probe pairs are preferably employed as FRET pairs. Moreover, the species-specific hybridization probe pairs of the invention are employed in analogy to the aforementioned for the specific detection of the mycobacterial species M. tuberculosis complex and M. avium, where in each case one hybridization probe partner (SEQ ID NO: 6 or the complement, or SEQ ID NO: 8 or the complement) is embodied as donor component (=anchor probe) which is preferably associated at the 3′-terminal nucleotide with a dye, preferably with a fluorescent dye, particularly preferably with fluorescein, and the respective other hybridization probe partner (SEQ ID NO. 7 or complement, or SEQ ID NO: 9 or complement) is embodied as acceptor component (=sensor probe) which is preferably associated at the 5′-terminal nucleotide with a further dye, preferably with a rhodamine derivative.

In preferred variants of the aforementioned embodiments, the rhodamine derivative is LightCycler Red 640; in further preferred variants of the aforementioned embodiment, the rhodamine derivative is LightCycler Red 705; in further preferred variants of the aforementioned embodiment, the rhodamine derivative is Cy5.

In one variant of the detection method of the invention, the same donor-acceptor dyes are used in each case for labelling the hybridization probe pairs, preferably fluorescein/LightCycler Red 640, in which case the melting curve analysis with the genus-specific probe pair for detecting mycobacteria is separated in time or space from the melting curve analysis with one of the species-specific hybridization probe pairs for detecting the M. tuberculosis complex or M. avium, in particular in parallel approaches in each case.

In a further variant of the detection method of the invention, different donor-acceptor dyes are used in each case for labelling the hybridization probe pairs, in which case the melting curve analysis with the genus-specific probe pair for detecting mycobacteria, which is preferably labelled with fluorescein/LightCycler Red 640, takes place together in time and space in one approach with melting curve analysis with one of the species-specific hybridization probe pairs, which is preferably labelled with fluorescein/LightCycler Red 705, for detecting the M. tuberculosis complex or M. avium, where the fluorescence of the genus-specific probe pair is recorded, preferably in the LightCycler™ system, with one photodetector channel and the fluorescence of a species-specific probe pair is recorded with the second photodetector channel.

In this connection, the present invention further relates to oligonucleotide primer pairs for multiplication of 16S rRNA fragments from extracted bacterial DNA for specific detection of mycobacteria and for differentiation of the Mycobacterium tuberculosis complex and Mycobacterium avium from other mycobacterial species in clinical material, where one Primer pair includes the nucleotide sequences SEQ ID NO: 2/SEQ ID NO: 3 or a pair of degenerate or mutated nucleotide sequences or fragments thereof. The degenerate or mutated nucleotide sequences have the property of in each case hybridizing with the sequences SEQ ID NO: 2/SEQ ID NO: 3, with preference in each case for a degree of homology of at least 90%, preferably of at least 95%, particularly preferably of at least 98%.

The invention also relates to a second primer pair with a primer with the nucleotide sequence SEQ ID NO: 4 or a degenerate or mutated nucleotide sequence or a fragment thereof, which hybridizes with the sequence SEQ ID NO: 4, with preference for a degree of homology of at least 90%, preferably of at least 95%, particularly preferably of at least 98%.

In this connection, the present invention further relates to oligonucleotide hybridization probe pairs for the specific detection of mycobacteria and for the differentiation of the Mycobacterium tuberculosis complex and Mycobacterium avium from other mycobacterial species in clinical material, selected from the group consisting of the nucleotide sequences SEQ ID NO: 10/SEQ ID NO: 11 or the pair of complementary sequences thereof, the nucleotide sequences SEQ ID NO: 6/SEQ ID NO: 7 or the pair of complementary sequences thereof and the nucleotide sequences SEQ ID NO: 8/SEQ ID NO: 9 or the pair of complementary sequences thereof.

In this connection, the present invention further relates to an artificial plasmid, control plasmid, preferably obtained by subcloning of the 16S rRNA gene into pGEM-T, which serves as internal control of the amplification (inhibition control) and of the specific detection of 16S rRNA fragments of mycobacteria, and comprises a nucleic acid sequence of the modified genus-specific region III of the 16S rRNA gene. The control plasmid of the invention is preferably derived through nucleotide exchange, nucleotide addition, nucleotide deletion and/or nucleotide inversion of at least one nucleotide, preferably of two nucleotides, from the wild-type nucleic acid sequence of the genus-specific region III of the 16S rRNA gene. In a further variant, the artificial plasmid includes the nucleotide sequence SEQ ID NO: 14 or SEQ ID NO: 15, in each of which one nucleotide is exchanged vis-à-vis the wild-type sequence. It has been found in this connection, particularly surprisingly, that through replacement of one nucleotide there is a reduction in the melting temperature of the genus-specific hybridization probes by approximately 1° C.

In a further particularly preferred variant, the artificial plasmid includes the nucleotide sequence SEQ ID NO: 16 or SEQ ID NO: 17, in each of which two nucleotides are exchanged vis-à-vis the wild-type sequence. It has been found in this connection, particularly surprisingly, that through the replacement of the two nucleotides there is a reduction in the melting temperature of the genus-specific hybridization probes by approximately 14.5° C. This advantageously permits simultaneous use of the modified 16S rRNA together with the wild-type 16S rRNA to be detected in one approach for the melting curve analysis, in particular by means of the genus-specific hybridization probe pair of the invention, because their melting temperatures are distinguishably far apart and thus the wild-type 16S rRNA fragment to be detected can be detected and identified uninfluenced by the internal standard.

In this connection, the present invention further relates to a diagnostic kit for the specific detection of a mycobacterial infection and of M. tuberculosis and M. avium in clinical material as claimed in the method of the invention, which includes at least one polymerase, at least one, preferably all, of the aforementioned primer pairs and at least one, preferably all, of the aforementioned hybridization probe pairs. It is preferred according to the invention for the diagnostic kit additionally to include at least one artificial control plasmid of the invention as internal standard.

Further advantageous embodiments are evident from the dependent claims.

### LIST OF REFERENCES

1. Bange, F. C., B. A. Brown, C. Smaczny, R. J. Wallace Jr, and E. C. Bottger. 2001. Lack of Transmission of Mycobacterium abscessus among Patients with Cystic Fibrosis Attending a Single Clinic. Clin. Infect. Dis. 32:1648-1650.
2. Chapin, K. and T. L. Lauderdale. 1997. Evaluation of a rapid air thermal cycler for detection of Mycobacterium tuberculosis. J. Clin. Microbiol. 35:2157-2159.
3. Desjardin, L. E., Y. Chen, M. D. Perkins, L. Teixeira, M. D. Cave, and K. D. Eisenach. 1998. Comparison of the ABI 7700 system (TaqMan) and competitive PCR for quantification of IS6110 DNA in sputum during treatment of tuberculosis. J. Clin. Microbiol. 36:1964-1968.
4. Holland, S. M. 2001. Nontuberculous mycobacteria. Am. J. Med. Sci. 321:49-55.
5. Kirschner, P., J. Rosenau, B. Springer, K. Teschner, K. Feldmann, and E. C. Bottger. 1996. Diagnosis of mycobacterial infections by nucleic acid amplification: 18-month prospective study. J. Clin. Microbiol. 34:304-312.
6. Kirschner, P., B. Springer, U. Vogel, A. Meier, A. Wrede, M. Kiekenbeck, F. C. Bange, and E. C. Bottger. 1993. Genotypic identification of mycobacteria by nucleic acid sequence determination: report of a 2-year experience in a clinical laboratory. J. Clin. Microbiol. 31:2882-2889.
7. Lindbrathen, A., P. Gaustad, B. Hovig, and T. Tonjum. 1997. Direct detection of Mycobacterium tuberculosis complex in clinical samples from patients in Norway by ligase chain reaction. J. Clin. Microbiol. 35:3248-3253.
8. McKinney, J. D., W. R. Jacobs, and B. R. Bloom. 1998. Persisting problems in tuberculosis, p. 51-139. In R. M. Krause (ed.), Emerging Infections. Academic Press, London.
9. Nivin, B., P. Nicholas, M. Gayer, T. R. Frieden, and P. I. Fujiwara. 1998. A continuing outbreak of multidrug-resistant tuberculosis, with transmission in a hospital nursery. Clin. Infect. Dis. 26:303-307.
10. Pfyffer, G. E. 1999. Nucleic acid amplification for mycobacterial diagnosis. J. Infect. 39 :21-26.
11. Taylor, M. J., M. S. Hughes, R. A. Skuce, and S. D. Neill. 2001. Detection of Mycobacterium bovis in Bovine Clinical Specimens Using Real-Time Fluorescence and Fluorescence Resonance Energy Transfer Probe Rapid-Cycle PCR. J. Clin. Microbiol. 39:1272-1278.
12. Tevere, V. J., P. L. Hewitt, A. Dare, P. Hocknell, A. Keen, J. P. Spadoro, and K. K. Young. 1996. Detection of Mycobacterium tuberculosis by PCR amplification with pan-Mycobacterium primers and hybridization to an M. tuberculosis-specific probe. J. Clin. Microbiol. 34:918-923.
13. Torres, M. J., A. Criado, J. C. Palomares, and J. Aznar. 2000. Use of real-time PCR and fluorimetry for rapid detection of rifampin and isoniazid resistance-associated mutations in Mycobacterium tuberculosis. J. Clin. Microbiol. 38:3194-3199.
14. World Health Organization. 2000. Drug-resistant strains of TB increasing worldwide (PR WHO/19; http://www.who.int).
15. Telenti, A., F. Marchesi, M. Balz, F. Bally, E. C. Bottger, and T. Bodmer. 1993. Rapid identification of mycobacteria to the species level by polymerase chain reaction and restriction enzyme analysis. J. Clin. Microbiol. 31:175-178.
16. Torrens, J. K., P. Dawkins, S. P. Conway, and E. Moya. 1998. Non-tuberculous mycobacteria in cystic fibrosis. Thorax 53:182-185.

The invention is explained in more detail by means of the sequence listing, which comprises Sequences Nos. 1 to 17, by means of FIGS. 1 to 4 and by means of Examples 1 to 8.

SEQ ID NO: 1—sense primer (forward primer) of the primer pair for amplifying a 1000 bp fragment of the 16S rRNA gene of mycobacteria, comprising the species-specific regions of the M. tuberculosis complex and of M. avium and the genus-specific region III of the genus Mycobacterium,

SEQ ID NO: 2—sense primer (forward primer) of the primer pair for amplifying a 300 bp fragment of the 16S rRNA gene of mycobacteria, comprising the species-specific regions of the M. tuberculosis complex and of M. avium,

SEQ ID NO: 3—antisense primer (reverse primer) of the primer pair for amplifying a 300 bp fragment of the 16S rRNA gene of mycobacteria, comprising the species-specific regions of the M. tuberculosis complex and of M. avium,

SEQ ID NO: 4—sense primer (forward primer) of the primer pair for amplifying a 100 bp fragment of the 16S rRNA gene of mycobacteria, comprising the genus-specific region III of the genus Mycobacterium,

SEQ ID NO: 5—antisense primer (reverse primer) a) of the primer pair for amplifying a 1000 bp fragment of the 16S rRNA gene of mycobacteria, comprising the species-specific regions of the M. tuberculosis complex and of M. avium and the genus-specific region III of the genus Mycobacterium, and b) the primer pair for amplifying a 100 bp fragment of the 16S rRNA gene of mycobacteria, comprising the genus-specific region III of the genus Mycobacterium,

SEQ ID NO: 6—antisense hybridization probe, in particular donor component, of the probe pair for detecting the species-specific region of the M. tuberculosis complex,

SEQ ID NO: 7—sense hybridization probe, in particular acceptor component, of the probe pair for detecting the species-specific region of the M. tuberculosis complex,

SEQ ID NO: 8—antisense hybridization probe, in particular donor component, of the probe pair for detecting the species-specific region of the M. avium,

SEQ ID NO: 9—sense hybridization probe, in particular acceptor component, of the probe pair for detecting the species-specific region of the M. avium,

SEQ ID NO: 10—antisense hybridization probe, in particular donor component, of the probe pair for detecting the genus-specific region III of the genus Mycobacterium,

SEQ ID NO: 11—sense hybridization probe, in particular acceptor component, of the probe pair for detecting the genus-specific region III of the genus Mycobacterium,

SEQ ID NO: 12—sense primer (forward primer) of the primer pair for amplifying the complete 16S rRNA gene (1523 bp) of mycobacteria,

SEQ ID NO: 13—antisense primer (reverse primer) of the primer pair for amplifying the complete 16S rRNA gene (1523 bp) of mycobacteria, SEQ ID NO: 14—modified sense primer (forward primer) for amplifying a complete control plasmid comprising a modified genus-specific region III of the 16S rRNA gene of mycobacteria,

SEQ ID NO: 15—modified antisense primer (reverse primer) for amplifying a complete control plasmid comprising a modified genus-specific region III of the 16S rRNA gene of mycobacteria,

SEQ ID NO: 16—modified sense primer (forward primer) for amplifying a further complete control plasmid comprising a further modified genus-specific region III of the 16S rRNA gene of mycobacteria,

SEQ ID NO: 17—modified antisense primer (reverse primer) for amplifying a further complete control plasmid comprising a further modified genus-specific region III of the 16S rRNA gene of mycobacteria.

The invention is explained in more detail by means of the following exemplary embodiments and the relevant figures:

The figures show:

FIG. 1: Diagrammatic representation of the 16S rRNA gene of mycobacteria (length: 1523 bp), of the position of the species-specific regions, “species(A)” and “species(B)”, and of the genus-specific regions “genus I”, “genus II” and “genus III”, and of the location and size of the fragments amplified by means of the primer pairs (1) and (5), (2) and (3), and (4) and (5).

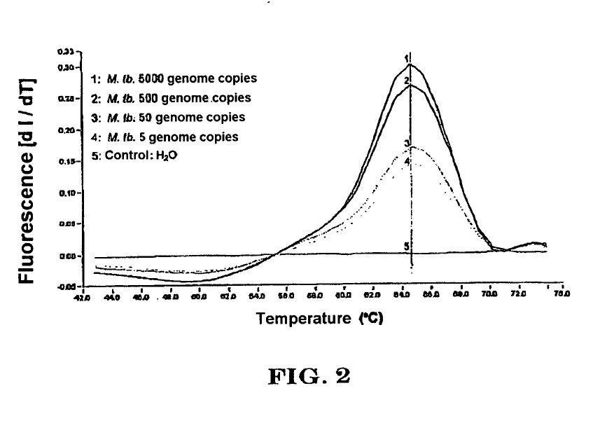

FIG. 2: Sensitivity of the species-specific detection of the M. tuberculosis complex: melting curves of the hybridization of the species-specific hybridization probes of the invention with the species-specific region of the M. tuberculosis complex in the amplified 1000 bp fragment of the 16S rRNA of mycobacteria.

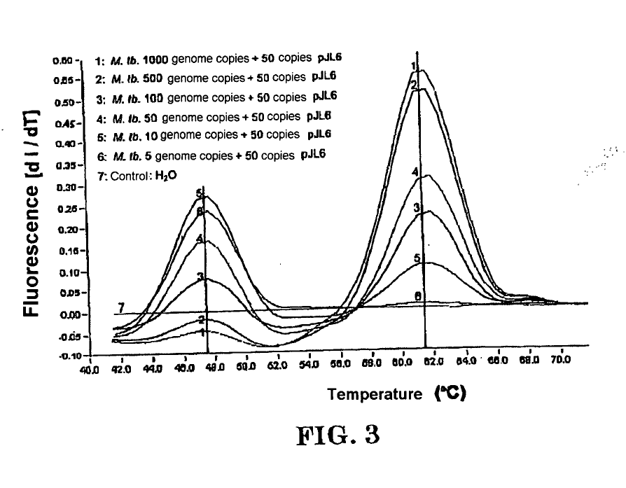

FIG. 3: Modified 16S rRNA fragment as internal standard (pJL6): melting curves of the hybridization of the genus-specific hybridization probes with the genus-specific region III and the modified genus-specific region III of the internal standard (pJL6) when the number of genome copies of the genus-specific region III to be detected differs.

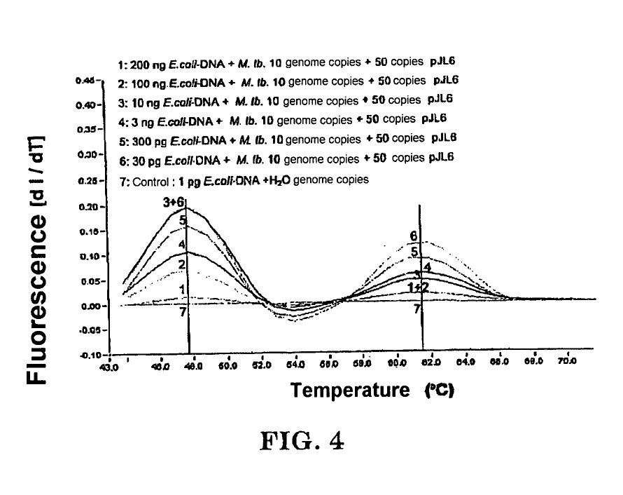

FIG. 4: Modified 16S rRNA fragment as internal standard (pJL6): melting curves of the hybridization of the genus-specific hybridization probes with the genus-specific region III and the modified genus-specific region III of the internal standard (pJL6) in the presence of a differing amount of “background” DNA from E. coli.

### EXAMPLE 1

a) DNA Isolation from Clinical Material

Microbial DNA is purified, i.e. extracted, from clinical samples consisting of sputum, bronchial lavage, gastric juice, urine, stool, liquor, bone marrow, blood or puncture biopsies in a manner known per se for example by means of a Qiamp™ MiniKit (from Qiagen, catalogue no. 51306). Chromosomal DNA is quantified by means of the PicoGreen™ system (from Molecular Probes).

Various numbers of genomic copies per preparation to be detected (see below) are obtained by molecular weight calculations and carrying out serial dilutions.

a′) DNA Isolation from Culture Isolates

Microbial DNA of various cultures of microorganisms, for example the organisms listed in Table 1, is isolated in particular for evaluating the detection methods of the invention. Microbial DNA is isolated from these cultures in particular for applying the detection method of the invention to the (broth) cultures, obtained from patients' samples, of microorganisms to be diagnosed.

The microbial DNA is then purified, i.e. extracted, in a manner known per se for example by means of a Qiamp™ MiniKit (from Qiagen, catalogue no. 51306). Chromosomal DNA is quantified in a manner known per se for example by means of the PicoGreen™ system (from Molecular Probes).

b) PCR Amplification

A mixture comprising the “LightCycler™ FastStart DNA Master Hybridisation Probes” mixture which is obtainable ready for use (catalogue no. 239272, from Roche Molecular Biochemicals) is chosen for amplification in an optimized LightCycler PCR (see Example 3).

The following reaction mixture is prepared for the LightCycler™ reaction:
Taq polymerase
Reaction buffer
Deoxynucleoside triphosphate mixture (dNTP)
3 mmol/l MgCl₂
primer pair a) or b) each primer: 18 pmol, equivalent to 1.1 μmol/l final concentration:
   a) primer pair SEQ ID NO: 1/SEQ ID NO: 5 or
   b) primer pair SEQ ID NO: 2/SEQ ID NO: 3 and primer pair SEQ ID NO: 4/SEQ ID NO: 5
The primer pair a) SEQ ID NO: 1/SEQ ID NO: 5 which amplifies a 1000 bp-long fragment of the 16S-rRNA, comprising the genus-specific region III and the species-specific regions, is employed according to the invention for the amplification.
Alternatively employed according to the invention are the two primer pairs b) simultaneously in one multiplex PCR mixture, in parallel mixtures or separated in time, where the primer pair comprising SEQ ID NO: 2/SEQ ID NO: 3 amplifies a 300 bp-long fragment of the 16S rRNA comprising the species-specific regions, and serves for the subsequent detection of the M. tuberculosis complex and/or of M. avium, and where the primer pair comprising SEQ ID NO: 4/SEQ ID NO: 5 amplifies a 100 bp-long fragment of the 16S rRNA comprising the genus-specific region III, and serves for the subsequent detection of a mycobacterial infection.
Oligonucleotide FRET probe pairs c) to e) per probe: 2 pmol, corresponding to a final concentration of 120 nmol/l:
   c) SEQ ID NO: 10/SEQ ID NO: 11 for detecting the genus-specific region III,
   d) SEQ ID NO: 6/SEQ ID NO: 7 for detecting the species-specific region of the M. tuberculosis complex,
   e) SEQ ID NO: 8/SEQ ID NO: 9 for detecting the species-specific region of M. avium
The probe pair c) SEQ ID NO: 10/SEQ ID NO: 11 or a complementary or mutated or degenerate pair thereof is employed according to the invention for detecting the genus-specific region III. If it is intended in the method of the invention merely to detect the M. tuberculosis complex in addition to the genus-specific detection, the probe pair d) SEQ ID NO: 6/SEQ ID NO: 7 or the equivalents thereof, i.e. mutated or complementary pairs thereof, is employed. If, on the other hand, it is intended merely to detect mycobacteria of the species M. avium in addition to the genus-specific detection, the probe pair e) SEQ ID NO: 8/SEQ ID NO: 9 or the equivalents thereof, i.e. mutated or complementary pairs thereof, is employed. If it is intended to detect together with the genus-specific region III both the M. tuberculosis complex and M. avium, in addition to probe pair c) correspondingly the two probe pairs d) and e) are employed.

This reaction mixture is introduced by pulse centrifugation into the glass capillaries of the LightCycler™ system, and the amplification is carried out on the “hot start” principle after initial denaturation at 95° C. for 10 minutes with the following steps:
1. Denaturatiion at 95° C. for 3 seconds
2. Primer hybridization at temperatures of 68° C. to 62° C. for 2 seconds (touch-down annealing)
3. Polymerisation at 72° C. for 40 seconds.

Steps 1 to 3 are performed cyclically a total of 50 times, with the hybridization in step 2 taking place at 68° C. for the first 5 cycles and the temperature being reduced in steps of 1° C. per cycle to 62° C. in the subsequent 6 cycles, and being carried out at 62° C. for the remaining cycles. The rate of temperature change is 20° C. per second in all the steps.

c) Detection and Melting Curve Analysis:

The amplified fragments are detected by using the FRET-labelled hybridization probe pairs employed in the reaction mixture, where in each case one hybridization probe partner (SEQ ID NO: 10, SEQ ID NO: 6 or SEQ ID NO: 8) is associated as donor component on the 3′-terminal nucleotide with fluorescein, and the respective other hybridization probe partner (SEQ ID NO: 11, SEQ ID NO: 7 or SEQ ID NO: 9) is associated as acceptor component on the 5′-terminal nucleotide with LightCycler™ Green 640.

The melting curve analysis which takes place during the detection starts with denaturation of the amplified fragments at 95° C. for 30 seconds, followed by hybridization with the aforementioned FRET pairs at 38° C. for 30 seconds. To determine the hybridization melting curve, the temperature is subsequently increased continuously from 38° C. to 80° C. at a rate of 0.2° C./sec with continuous recording of the fluorescence emitted by the FRET pairs. The fluorescence signal is analyzed by employing the LightCycler Run Profile programme in version 3.5.3, with the amplification of the F2 channel of the photometric detector of the LightCycler™ system being set automaticically.

### EXAMPLE 2

### Artificial Plasmid as Internal Standard

a) Preparation of a Control Plasmid (According to the Invention)

In order have an internal standard available for checking the successful selective amplification of the desired fragments of the 16S rRNA gene of mycobacteria as in Example 1 (inhibition control), initially the complete mycobacterial 16S rRNA gene (1523 bp) is amplified with a PCR primer pair where the sense primer consists of the nucleotide sequence SEQ ID NO: 12 and the antisense primer consists of the nucleotide sequence SEQ ID NO: 13. For the amplification, 40 cycles of the following steps are carried out:
1. Denaturation at 95° C.
2. Primer hybridization at 56.5° C.
3. Polymerization at 72° C.

The amplified fragments are then subcloned in a manner known per se for example into pGEM-T (from Promega).

In order to be able to employ the artificial plasmid as internal standard, the genus-specific region III, present in the artificial plasmid (=pIJ6), of the 16S rRNA should differ by at least one point mutation from the wild-type nucleotide sequence of the genus-specific region III. In order to introduce at least one specific point mutation into the genus-specific region III of the 16S rRNA fragment, the plasmids containing the subcloned fragments are multiplied by employing modified primer pairs in which in each case one or two nucleotides in the nucleotide sequences have been exchanged vis-à-vis the wild-type nucleotide sequences.

For this purpose, the following primer pairs derived from the wild-type sequence of the genus-specific region III, in particular from the region binding the acceptor component of the FRET pair of the invention, SEQ ID NO: 11, are used:

a) Exchange of One Nucleotide:

| | |
|---|---|
| forward primer: | |
| 5′-GGC TTG ACA TGC ACA GGA CGC-3′ | (SEQ ID NO:14) |
| reverse primer: | |
| 5′-GCG TCC TGT GCA TGT CAA GCC-3′ | (SEQ ID NO:15) |

b) Exchange of Two Nucleotides:

| | |
|---|---|
| forward primer: | |
| 5′-GGT TTG ACA TAC ACT GGA CGC-3′ | (SEQ ID NO:16) |
| reverse primer: | |
| 5′-GCG TCC AGT GTA TGT CAA ACC-3′ | (SEQ ID NO:17) |

In the nucleotide sequences of the invention of the aforementioned modified primer pairs, in each case the underlined nucleotide was replaced vis-à-vis the wild-type sequence of the genus-specific region III.

Multiplication of the control plasmids with the modified primer pairs is in each case carried out in a “long range” PCR with a Pfu polymerase, type: “Pfu Turbo “Hot Start” DNA” (from Stratagene) in a manner known per se in a Hot Start PCR method with in each case 18 cycles of the following steps:
1. Denaturation at 95° C.
2. Primer hybridization at 50° C.
3. Polymerization at 68° C.
   Results:

a) The resulting amplicons are additionally purified on a gel, and the point mutations can be subsequently be confirmed by sequencing.

b) Replacement of the nucleotide ‘T’ by the nucleotide ‘C.’ at the 3′ end of the region, which binds the acceptor component of the FRET pair, of the genus-specific region III leads to a reduction of approximately 1° C. in the melting temperature.

c) Replacement at the 5′ end of the nucleotide ‘G’ by the nucleotide ‘A’ and, four nucleotides distant from this, of the nucleotide ‘A’ by the nucleotide ‘T’ results in a significant reduction of about 14.5° C. in the melting temperature.

It is found to be particularly surprising that a modification comprising two nucleotides of the region, which binds the acceptor component of the FRET pair, of the genus-specific region III is sufficient to differentiate a differentiation of the 16S rRNA modified in this way and present in an artificial plasmid from the wild-type 16S rRNA. The use of the modified 16S rRNA fragment of the invention as internal standard present in an artificial plasmid in the method of the invention as described in Example 1 is thus particularly advantageous for checking the amplification and for verifying the detection of a mycobacterial infection.

b) Suitability of the Control Plasmid when the Amount of Sample Genome is Small

In a further experiment, firstly a control plasmid is obtained, as described under a), which comprises a 16S rRNA fragment modified by exchange of two nucleotides through use of the primers of the invention SEQ ID NO: 16 and SEQ ID NO: 17. In each case 50 copies of the control plasmid are mixed as internal standard with various numbers of gene copies of the 16S rRNA gene of M. tuberculosis and subjected to the detection method described in Example 1.

Result:

In the presence of 50 copies of the internal standard it was possible to detect unambiguously by means of melting curve analysis as few as 10 gene copies of the 16S rRNA fragment of M. tuberculosis. Although the fluorescence signal obtained when the number of gene copies is 5 cannot be analyzed by simple statistical means, a distinct peak is detectable at the appropriate melting temperature in the family of melting curves in FIG. 3.

The use of this internal standard is thus possible even when it is to be expected that the number of gene copies to be detected in the clinical material is an order of magnitude smaller than the number of plasmid copies employed as internal standard.

c) Suitability of the Control Plasmid in the Presence of “Background” DNA

In a further experiment, firstly a control plasmid is obtained, as described under a), which comprises a 16S rRNA fragment modified by exchange of two nucleotides through use of the primers of the invention SEQ ID NO: 16 and SEQ ID NO: 17. In each case 50 copies of the control plasmid and 10 gene copies of the 16S rRNA gene of M. tuberculosis are mixed with various amounts of “background” E. coli DNA in the range from 1 pg to 200 ng and then subjected to the detection method described in Example 1.

Result:

In the presence of to 200 ng of foreign DNA, so-called “background” DNA, per mixture it was still possible to detect 10 gene copies of the 16S rRNA fragment of M. tuberculosis unambiguously by means of melting curve analysis (FIG. 4).

The detection method of the invention, especially using this internal standard, is thus possible even if it is to be expected that a large amount of foreign DNA, “background” DNA, is present in the isolated clinical material.

### EXAMPLE 3

### Assessment of the Efficiency of the Detection Method of the Invention in the LightCycler™ System

The amplification and hybridization based on the LightCycler™ system (see Example 1) is investigated by using genomic DNA of the bacterial strain Mycobacterium bovis BCG of the M. tuberculosis complex as “template”.

Various numbers of genomic copies per PCR reaction are obtained by molecular weight calculations and carrying out serial dilutions.

The following parameters were employed for the combined amplification and hybridization reactions:
MgCl₂ concentration 3 mmol/l
Primer concentration: 18 pmol per reaction, equivalent to a final concentration of 1.1 μmol/l
“Hot Start” Taq polymerase of the type “LightCycler™ FastStart DNA Master SYBR GreenI” (catalogue no. 3003230)
Polymerization time: 40 seconds
Hybridization time: 3 seconds
Hybridization temperature: 62° C., with stepwise reduction starting from 68° C. after 5 cycles at 1° C. per cycle.
   Results:

A number of 5 genome copies can be detected reproducibly in serial dilutions.

Both the use of a “Hot Start” polymerase and the cycle-wise reduction in the hybridization temperature prevents the formation of primer dimers and the increases the sensitivity of the amplification.

### EXAMPLE 4

### Specific Amplification of Mycobacterial 16S rRNA Fragments (According to the Invention)

In a first mixture as in Example 1 and using the appropriate primer pairs of the invention a fragment, which is 100 bp long in each case, of the genus-specific region III (SEQ ID NO: 4/SEQ ID NO: 5) and a 300 bp-long fragment of the species-specific regions (SEQ ID NO: 2/SEQ ID NO: 3) is amplified.

In a second mixture as in Example 1, a 1000 bp-long fragment which comprises both the genus-specific region III and the two species-specific regions is amplified with the appropriate primer pair (SEQ ID NO: 1/SEQ ID NO: 5).

Subsequently, the sensitivity of the detection method of the invention as described in Example 1 is tested.

Result:

It surprisingly emerges that the 1000 bp-long fragment has the same sensitivity of detection vis-à-vis the two 100 bp- and 300 bp-long fragments. This result contrasts with the prejudice in the art that the high CG nucleotide content in the 16S rRNA gene of mycobacteria leads to nonspecific interference with the detection method described in Example 1 in a LightCycler™ system.

### EXAMPLE 5

### Sensitivity of the Detection Method of the Invention

The sensitivity is checked by using the 1000 bp fragment amplified with the primer pair SEQ ID NO: 1/SEQ ID NO: 5 in the detection method of the invention (Example 1).

a) Sensitivity of Detection of the Genus-Specific Region III

Firstly, serial dilutions, containing in each case 5, 50, 500 or 5000 genome copies, of the genome of M. bovis BCG from the M. tuberculosis complex are prepared.

In further mixtures, serial dilutions of further tuberculous and non-tuberculous mycobacterial species are investigated.

Results:

a) A melting curve can be determined unambiguously even with 5 genome copies, and the melting point of the genus-specific region III is at least 55° C. for all mycobacterial species. The melting point is 55° C. for M. chelonae, and is 61.5° C. for all other mycobacterial species.

b) It is additionally evident that unambiguous detection of the genus-specific region III is possible with a number of 5 genome copies for a large number of other mycobacterial species, for example of M. tuberculosis, M. bovis, M. avium, M. intracellulare, M. paratuberculosis, M. kansasii, M. marinum, M. abcessus, M. fortuitum.

b) Sensitivity of the Detection of M. tuberculosis

Serial dilutions of the M. tuberculosis genome in each case comprising 5, 50, 500 or 5000 copies is prepared as in a).

Result:

The melting curve of the species-specific region of M. tuberculosis can be determined unambiguously even with 5 genome copies (FIG. 2). In this case, the melting point is found to be 64° C. (Table 1).

c) Sensitivity of the Detection of M. avium

Serial dilutions of the genome of M. avium are prepared as in a) and b), and a melting curve analysis of the amplified fragments is carried out with the appropriate hybridization probe pairs.

Result:

Melting curve analysis of the amplicon is possible even starting from 5 genome copies, with a melting point of 61.0° C. being found (Table 1).

### EXAMPLE 6

### Specificity of the Detection Method of the Invention

a) Specificity of the Amplification Primers

2.5 ng of genomic DNA, equivalent to 500,000 genome copies, is extracted in each case per mixture from the microorganisms from Table 1 and employed in the detection method of the invention.

As in Example 5, a 1000 bp-long fragment of the 16S rRNA gene is amplified with the genus-specific primer pair SEQ ID NO: 1/SEQ ID NO: 5.

Results:

a) Amplification takes place with all the mycobacterial species investigated.

b) The primer pair employed shows almost complete genus specificity for mycobacteria: selected from the large number of different bacterial and fungal microorganisms, amplification of the 16S rRNA gene takes place only for the genus Corynebacterium.

b) Specificity of the Genus-Specific Detection

A melting curve analysis of the in amplified fragments by means of the hybridization probe pair SEQ ID NO: 10 /SEQ ID NO: 11 of the invention, specifically for the genus-specific region III, is carried out.

Results (Table 1):

a) All mycobacterial species exhibit a melting point of at least 55° C.

b) The melting point of M. chelonae is 55° C., and is 61.5° C. for all other mycobacterial species.

c) The melting point in the case of amplified 16S rRNA fragments of the genus Corynebacterium is 43° C., or no hybridization signal whatsoever can be detected.

d) All mycobacterial species can be identified unambiguously vis-à-vis other microorganisms by means of the genus-specific detection method of the invention.

c) Specificity of the Detection of the M. tuberculosis Complex

Firstly, as in a), in each case 2.5 ng of genomic DNA of the microorganisms listed in Table 1 are employed, and in each case a 1000 bp-long fragment is amplified.

Subsequently, a melting curve analysis of the amplicons is carried out with the species-specific hybridization probe pair SEQ ID NO: 6/SEQ ID NO: 7.

Result:

Whereas the melting temperature is 64° C. for all of the species of the at M. tuberculosis complex, the melting temperature for all non-tuberculous pathogens is between 43.5° C. and 54° C., if a hybridization signal can in fact be detected (Table 1).

It is possible by means of the detection method of the invention for tuberculous pathogens to be detected selectively vis-à-vis all other non-tuberculous pathogens.

d) Specificity of the Detection of M. avium

Firstly, as in a), in each case 2.5 ng of genomic DNA of the microorganisms listed in Table 1 are employed, and in each case a 1000 bp-long fragment is amplified.

Subsequently, a melting curve analysis of the amplicons is carried out with the species-specific hybridization probe pair SEQ ID NO: 8/SEQ ID NO: 9.

Result:

Whereas the melting temperature is 61° C. in all cases with M. avium, the melting temperature for all other pathogens is between 43.5° C. and 54° C., if a hybridization signal can in fact be detected (Table 1).

It is possible by means of the detection method of the invention for the non-tuberculous pathogen M. avium to be detected selectively vis-à-vis all other pathogens.

### EXAMPLE 7

### Comparative Example on the Specificity of the Genus-Specific Detection by Means of the Genus-Specific Region II (“Genus II”)

In contrast to Example 6 b) of the invention above, the hybridization probes which are known in the art and which are selective for the genus-specific region II (see FIG. 1; ‘genus II’) are used for the melting curve analysis of the 1000 bp fragment, amplified as in 6 a), of the 16S rRNA gene of mycobacteria from Table 1.

Table 1 shows the results of the melting curve analysis: whereas a large proportion of the investigated mycobacterial species has a melting point of 60.5° C., the melting point of the mycobacterial species M. triviale, M. chitae, M. xenopi and M. agri is reduced compared with other mycobacterial species (Table 1). In addition, amplicons were detected also with the genus Corynebacterium.

Result:

The melting points found for M. triviale, M. chitae, M. xenopi, M. agri and all other mycobacterial species cannot be separated statistically from the melting points found. for the Corynebacterium species. Unambiguous detection of a mycobacterial infection vis-à-vis for example an infection with Corynebacteria is not possible by means of the genus-specific region II.

### EXAMPLE 8

### Quantitative Determination of the Content of Mycobacteria in Clinical Samples (According to the Invention)

In the FRET system employed according to the invention, under certain conditions the degree of fluorescence in this wavelength range is a function of the amount of DNA present and detected in the sample. Quantitative measurements of the amount of amplified DNA fragments are possible by the FRET system if the selected hybridization probes bind to the amplified fragments quantitatively, i.e. stoichiometrically.

For quantification of target DNA, the latter is compared with a known DNA concentration of a standard. The cloned 16S rRNA gene im pGEM-T or else the control plasmid which can be employed according to the invention (pIJ6) is suitable for this purpose.

In this method, the fluorescence is measured after each of the total of 50 amplification cycles. If the concentration of the standard is high, a fluorescence signal appears for example after only 20 cycles. A low concentration leads to a signal only after for example 35 cycles.

The standard is measured on the one hand separately from the samples to be investigated, using at least five different concentrations in order to construct a standard curve. If the plasmid pIJ6 is used, it is possible in each case to use one concentration of the standard in each sample investigated. Differentiation between standard and target DNA takes place via the difference in melting point. The standard serves in particular simultaneously as control that the amplification was not inhibited by interfering factors (inhibition control).

The FRET-labelled hybridization probe pairs employed in the reaction mixture are used to detect the amplified fragments. The amplification reaction proceeds as in Example 1, measuring the fluorescence after each amplification step. The optimal annealing temperature is 62° C. The melting point analysis is carried out unimpaired after completion of the amplification reaction as in Example 1 c).

TABLE 1

| | Melting temperature with | | | | |
|---|---|---|---|---|---|
| | hybridization probes specific for | | | | |
| | according to the invention | | | | Comparative |
| | | Genus | Mycobacterium | | Genus |
| Microorganism | A* | III | tuberculosis | avium | II |

I. Mycobacterium tuberculosis complex

| | | | | | |
|---|---|---|---|---|---|
| M. tuberculosis H37Rv | + | 61.5° C. | 64° C. | 54° C. | 60.5° C. |
| (ATCC 35712) | | | | | |
| M. bovis | + | 61.5° C. | 64° C. | 54° C. | 60.5° C. |
| M. bovis BCG Pasteur | + | 61.5° C. | 64° C. | 54° C. | 60.5° C. |

II. non-tuberculous mycobacteria

| | | | | | |
|---|---|---|---|---|---|
| M. avium (ATCC35712) | + | 61.5° C. | 43.5° C. | 61° C. | 60.5° C. |
| M. paratuberculosis | + | 61.5° C. | 43.5° C. | 61° C. | 60.5° C. |
| M. intracellulare | + | 61.5° C. | — | 51° C. | 60.5° C. |
| M. kansasii (DSMZ 44162) | + | 61.5° C. | 50° C. | 48° C. | 60.5° C. |
| M. gastri (DSMZ 43505) | + | 61.5° C. | 50° C. | 48° C. | 60.5° C. |
| M. abscessus (ATCC 19977) | + | 61.5° C. | — | — | 60.5° C. |
| M. chelonae (ATCC 35752) | + | 55° C. | — | — | 60.5° C. |
| M. celatum (ATCC 58131) | + | 61.5° C. | — | 44° | 60.5° C. |
| M. farcinogenes (ATCC 35753) | + | 61.5° C. | 50° C. | 48° C. | 60.5° C. |
| M. hamophilum (ATCC 29548) | + | 61.5° C. | 50° C. | 48° C. | 60.5° C. |
| M. malmoense (ATCC 27046) | + | 61.5° C. | — | 43° C. | 60.5° C. |
| M. marinum (ATCC 927) | + | 61.5° C. | 45° C. | 48° C. | 60.5° C. |
| M. scrofulaceum (ATCC 19981) | + | 61.5° C. | 50° C. | 48° C. | 60.5° C. |
| M. shimoidei (ATCC 27962) | + | 61.5° C. | 50° C. | 48° C. | 60.5° C. |
| M. xenopi | + | 61.5° C. | 54° C. | 47° C. | 59° C. |
| M. simile | + | 61.5° C. | 50° C. | 48° C. | 60.5° C. |
| M. agri (ATCC 27406) | + | 61.5° C. | — | 43° C. | 53° C. |
| M. triviale (ATCC 23292) | + | 61.5° C. | 48° C. | 49° C. | 53° C. |
| M. fortuitum | + | 61.5° C. | 45° C. | 47° C. | 60.5° C. |
| M. chitae (ATCC 19627) | + | 61.5° C. | 51° C. | 51° C. | 56.5° C. |
| M. duvalii (ATCC 43910) | + | 61.5° C. | 43° C. | 45° C. | 60.5° C. |
| M. neoaurum (ATCC 25795) | + | 61.5° C. | 48° C. | 53° C. | 60.5° C. |
| M. phlei (ATCC 11758) | + | 61.5° C. | — | 44° C. | 60.5° C. |
| M. rhodesiae (ATCC 27024) | + | 61.5° C. | 52° C. | 52° C. | 60.5° C. |
| M. smegmatis | + | 61.5° C. | — | — | 60.5° C. |
| M. senegalense (ATCC 33027) | + | 61.5° C. | 46° C. | 48° C. | 60.5° C. |
| M. porcinum (ATCC 33776) | + | 61.5° C. | 49° C. | 51° C. | 60.5° C. |
| M. gordonae (DSMZ 44160) | + | 61.5° C. | — | 42.5° C. | 60.5° |
| M. szulgai (ATCC 35799) | + | 61.5° C. | 51° C. | 42.5° C. | 60.5° |
| M. genavense | + | 61.5° C. | 51° C. | 50° C. | 60.5° |

III. non-mycobacterial actinomyces

| | | | | | |
|---|---|---|---|---|---|
| Nocardia farcinica (ATCC 3318) | − | | | | |
| Nocardia brevicatena | − | | | | |
| (ATCC 15333) | | | | | |
| Streptomyces griseus | − | | | | |
| Rhodococcus equi | − | | | | |
| Coryneb. pseudodiphteriticum | + | 43° C. | 44° C. | 44° C. | 47° C. |
| (ATCC 10700) | | | | | |
| Coryneb. jeikeium | + | 44° C. | 44° C. | 44° C. | 47° C. |
| Coryneb. xerosis (ATCC 373) | + | | | | 47° C. |

IV. Gram-positive bacteria

| | |
|---|---|
| Bacillus subtilis (ATCC 6633) | − |
| Bacillus cereus | − |
| Staphylococcus aureus | − |
| (ATCC 25923) | |
| Staphylococcus epidermidis | − |
| (ATCC 12228) | |
| Streptococcus pneumoniae | − |
| (ATCC 49619) | |
| Listeria monocytogenes | − |
| (ATCC 19115) | |
| Enterococcus faecalis | − |
| (ATCC 29212) | |

V. Gram-negative bacteria

| | |
|---|---|
| Proteus mirabilis (ATCC 14153) | − |
| Escherchia coli (ATCC 25922) | − |
| Salmoneila typhimurium | − |
| (ATCC 14028) | |
| Shigella sonnei (ATCC 25930) | − |
| Klebsiella pneumoniae | − |
| (ATCC 10031) | |
| Pseudomonas aeruginosa | − |
| (ATCC 27853) | |
| Moraxella catarrhalis | − |
| (ATCC 19115) | |

VI. Fungi

| | |
|---|---|
| Candida albicans | − |
| Candida glabrata | − |
| Candida crusei | − |
| Aspergillus fumigatus | − |
| Fusarium | − |

*A: Amplification of the 16S rRNA fragment with the genus-specific primers of the invention
+: Amplification took place
−: Amplification did not take place

1. A method for the joint and in each case specific detection of a mycobacterial infection, of the Mycobacterium tuberculosis complex and/or of Mycobacterium avium in clinical material comprising the steps of
a) extraction of microbial DNA from clinical material,
b) amplification of at least one fragment of the 16S rRNA gene from the extracted DNA by means of a primer pair including the nucleotide sequences SEQ ID NO: 1/SEQ ID NO: 5 or by means of two primer pairs, where one primer pair includes the nucleotide sequence SEQ ID NO: 2/SEQ ID NO: 3 and the other primer pair includes the nucleotide sequences SEQ ID NO: 4/SEQ ID NO: 5,
c) detection of the genus-specific region of the amplified 16S rRNA fragment of mycobacteria by means of a pair of labelled hybridization probes, where the pair includes the nucleotide sequences SEQ ID NO: 10/SEQ ID NO: 11,
c) detection of the species-specific region of the amplified 16S rRNA fragment of mycobacteria by means of a pair of labelled hybridization probes, where the pair includes the nucleotide sequences SEQ ID NO: 6/SEQ ID NO: 7 or the complementary sequences thereof for detecting the Mycobacterium tuberculosis complex, and where the pair includes the nucleotide sequences of SEQ ID NO: 8/SEQ ID NO: 9 or the complementary sequences thereof for detecting Mycobacterium avium, and
e) where the joint, in each case specific detection of mycobacteria and of the Mycobacterium tuberculosis complex and/or of Mycobacterium avium takes place during the detection as in steps c) and d) by means of melting curve analysis.

2. The method according to claim 1, where the extracted microbial DNA is mixed in step a) of the method with at least one artificial plasmid which serves as internal standard, where the artificial plasmid includes a genus-specific region III of the 16S rRNA of mycobacteria or parts thereof, which has been modified in its nucleotide sequence, and where a melting curve analysis is carried out for specific detection of the amplified 16S rRNA fragments of mycobacteria and of the modified 16S rRNA fragments of the artificial plasmid.

3. The method according to claim 1, where the extracted microbial DNA is divided and a first portion thereof is further treated in the method specified in steps a) to e) of claim 1, and a second portion is subjected to a parallel method comprising the steps of
a′) mixing of the extracted microbial DNA with at least one artificial plasmid as internal standard, where the artificial plasmid includes a genus-specific region III of the 16S rRNA of mycobacteria or parts thereof, which has been modified in its nucleotide sequence,
b′) amplification of the 16S rRNA fragment by means of at least one primer pair selected from the group including the nucleotide sequence pairs SEQ ID NO: 1/SEQ ID NO: 5 and SEQ ID NO: 4/SEQ ID NO: 5,
c′) detection of the amplified 16S rRNA fragments by means of a pair of labelled hybridization probes which hybridize with the genus-specific region III of the 16S rRNA fragment of mycobacteria, where the pair includes the nucleotide sequences SEQ ID NO: 10/SEQ ID NO: 11 or the complementary sequences thereof, and
d′) where a melting curve analysis takes place for the specific detection of the amplified 16S rRNA fragments of mycobacteria and of the modified 16S rRNA fragments of the at least one artificial plasmid during the detection as in step c′).

4. The method according to any of the preceding claims, where the amplification of the gene fragments is carried out by means of the polymerase chain reaction (PCR).

5. The method according to claim 4, where the polymerase chain reaction (PCR) is carried out as real-time PCR, preferably by means of the LightCycler™ system.

6. The method according to any of the preceding claims, where the detection takes place during or after the amplification of the 16S rRNA fragments.

7. The method according to any of the preceding claims, where the detection takes place by means of real-time PCR, particularly preferably by means of the LightCycler™ system.

8. The method according to any of the preceding claims, where the detection is carried out by means of fluorescence detection, and where the labelled hybridization probe pairs are configured as fluorescence resonance energy transfer pair.

9. The method according to any of the preceding claims, where the melting curve analysis takes place following the amplification of the 16S rRNA fragments by means of real-time PCR, preferably by means of the LightCycler™ system.

10. The method according to claim 8 or 9, where the detection is carried out as quantitative measurement.

11. The method according to any of the preceding claims, where the clinical material is selected from the group of clinical samples consisting of sputum, bronchial lavage, gastric juice, urine, stool, liquor, bond marrow, blood and biopsies.

12. An oligonucleotide primer pair which comprises the nucleic acids with the nucleotide sequences SEQ ID NO: 2 and SEQ ID NO: 3.

13. An oligonucleotide primer which comprises the nucleic acids with the nucleotide sequence SEQ ID NO: 4.

14. An oligonucleotide hybridization probe pair which comprises the nucleic acids with the nucleotide sequences SEQ ID NO: 6 and SEQ ID NO: 7 or with the complementary sequences thereof.

15. An oligonucleotide hybridization probe pair which comprises the nucleic acids with the nucleotide sequences SEQ ID NO: 8 and SEQ ID NO: 9 or with the complementary sequences thereof.

16. An oligonucleotide hybridization probe pair which comprises the nucleic acids with the nucleotide sequences SEQ ID NO: 10 and SEQ ID NO: 11 or with the complementary sequences thereof.

17. An artificial plasmid which can be employed as internal control of the amplification and of the detection of 16S rRNA fragments of mycobacteria, including modified sequences of the genus-specific region III of the 16S rRNA gene.

18. An artificial plasmid according to claim 17, where the artificial plasmid comprises at least one nucleotide exchange, addition, deletion and/or inversion vis-à-vis the wild-type sequence of the genus-specific region III of the 16S rRNA gene.

19. An artificial plasmid as claimed in claim 17 or 18, including the nucleotide sequence SEQ ID NO: 14 or SEQ ID NO: 15.

20. An artificial plasmid as claimed in claim 17 or 18, including the nucleotide sequence SEQ ID NO: 16 or SEQ ID NO: 17.

21. A diagnostic kit for the specific detection of a mycobacterial infection and of the Mycobacterium tuberculosis complex and of Mycobacterium avium in clinical material by the method according to any of claims 1 to 11 including:
a) at least one polymerase,
b) at least one primer pair according to claim 12,
c) at least one primer pair comprising a primer according to claim 13 which amplifies the genus-specific region III of mycobacteria.
d) a hybridization probe pair according to claim 16 for the detection of the genus-specific region III,
e) at least one hybridization probe pair according to claims 14 to 15 for the detection of the species-specific regions.

22. A diagnostic kit according to claim 21, comprising an artificial plasmid according to any of claims 17 to 20.


SEQUENCE LISTING (17 sequences)

SEQ ID NO: 1 - DNA, 21 bp, Mycobacterium
gagtttgatcctggctcagga

SEQ ID NO: 2 - DNA, 18 bp, Mycobacterium
gataagcctgggaaactg

SEQ ID NO: 3 - DNA, 18 bp, Mycobacterium
ctaccgtcaatccgagag

SEQ ID NO: 4 - DNA, 18 bp, Mycobacterium
ggcggagcatgtggatta

SEQ ID NO: 5 - DNA, 20 bp, Mycobacterium
tgcacacaggccacaaggga

SEQ ID NO: 6 - DNA, 20 bp, Mycobacterium
cgcgggctcatcccacaccg

SEQ ID NO: 7 - DNA, 22 bp, Mycobacterium
taaagcgctttccaccacaaga

SEQ ID NO: 8 - DNA, 20 bp, Mycobacterium
cgcgggcccatcccacaccg

SEQ ID NO: 9 - DNA, 21 bp, Mycobacterium
aaaagctttccaccagaagac

SEQ ID NO: 10 - DNA, 23 bp, Mycobacterium
gcaacgcgaagaaccttacctgg

SEQ ID NO: 11 - DNA, 18 bp, Mycobacterium
tttgacatgcacaggacg

SEQ ID NO: 12 - DNA, 21 bp, Mycobacterium
gagtttgatcctggctcagga

SEQ ID NO: 13 - DNA, 20 bp, Mycobacterium
aaggaggtgatccagccgca

SEQ ID NO: 14 - DNA, 21 bp, Mycobacterium
ggcttgacatgcacaggacgc

SEQ ID NO: 15 - DNA, 21 bp, Mycobacterium
gcgtcctgtgcatgtcaagcc

SEQ ID NO: 16 - DNA, 21 bp, Mycobacterium
ggtttgacatacactggacgc

SEQ ID NO: 17 - DNA, 21 bp, Mycobacterium
gcgtccagtgtatgtcaaacc